(19) United States
(12) Patent Application Publication
(10) Pub. No.: US 2020/0230071 A1
(43) Pub. Date: Jul. 23, 2020

(54) METHODS OF PRODUCING HOLLOW METAL NANOSPHERES

(71) Applicant: The Regents of the University of California, Oakland, CA (US)

(72) Inventors: Sarah Lindley, Santa Cruz, CA (US); Jin Zhang, Santa Cruz, CA (US)

(21) Appl. No.: 16/746,306

(22) Filed: Jan. 17, 2020

Related U.S. Application Data
(60) Provisional application No. 62/794,438, filed on Jan. 18, 2019.

(51) Int. Cl. A61K 9/51; A61K 41/00; B22F 1/00; B22F 9/16

(52) U.S. Cl. CPC A61K 9/5192; A61K 9/5115; A61K 41/0052; B22F 2301/255; B22F 9/16; B22F 2001/0029; B22F 1/0018

(57) ABSTRACT

Provided are methods of producing hollow metal nanospheres (HMNs) having a pre-selected surface rugosity. The methods include combining in a galvanic exchange reaction at a selected pH: a solution comprising cobalt-based nanoparticle (CoₓB_{y} NP) scaffolds; and a solution comprising a metal, to produce CoₓB_{y} NP core/metal shell structures. The methods further include oxidizing the CoₓB_{y} NP cores of the CoₓB_{y} NP core/metal shell structures to produce HMNs having the pre-selected surface rugosity, where the pH of the galvanic exchange reaction is selected to produce the pre-selected surface rugosity of the HMNs. Also provided are HMNs produced according to the methods, as well as methods of using the HMNs. Compositions and kits that find use, e.g., in practicing the methods of the present disclosure, are also provided.

### CROSS-REFERENCE TO RELATED APPLICATIONS

This application claims the benefit of U.S. Provisional Patent Application No. 62/794,438, filed Jan. 18, 2019, which application is incorporated herein by reference in its entirety.

### STATEMENT OF GOVERNMENTAL SUPPORT

This invention was made with government support under grant number NNX15AQ01A awarded by the National Aeronautics and Space Administration (NASA). The government has certain rights in the invention.

### INTRODUCTION

Plasmonic nanostructures are attractive components in a variety of applications because of their tunable optical and electronic properties, particularly due to their structure-dependent surface plasmon resonance (SPR) enabling strong absorption and scattering of specific wavelengths of incident light. For biomedical applications in particular, much effort has been focused on fabricating particles with SPR frequencies in the near-infrared (NIR) to be compatible with the transparency window of biological tissue. This effort has resulted in a recent proliferation of NIR gold nanostructures in the literature, including rods, bipyramids, octahedra, shells, cages, and hollow nanospheres. Hollow structures have proven to be especially advantageous as the core provides an additional structural parameter by which to modify the optical properties. For instance, the SPR frequency of the hollow gold nanosphere (HGN) may be red-shifted by either an increase in outer diameter or a decrease in shell thickness. This enables a broad range of biocompatible sizes to be prepared with NIR SPR frequencies. In addition to greater tunability, hollow structures have exhibited better photophysical performance than their solid counterparts in applications spanning drug delivery, sensing, and catalysis.

### SUMMARY

Provided are methods of producing hollow metal nanospheres (HMNs) having a pre-selected surface rugosity. The methods include combining in a galvanic exchange reaction at a selected pH: a solution comprising cobalt-based nanoparticle (CoₓB_{y} NP) scaffolds; and a solution comprising a metal, to produce CoₓB_{y} NP core/metal shell structures. The methods further include oxidizing the CoₓB_{y} NP cores of the CoₓB_{y} NP core/metal shell structures to produce HMNs having the pre-selected surface rugosity, where the pH of the galvanic exchange reaction is selected to produce the pre-selected surface rugosity of the HMNs. Also provided are HMNs produced according to the methods, as well as methods of using the HMNs. Compositions and kits that find use, e.g., in practicing the methods of the present disclosure, are also provided.

### BRIEF DESCRIPTION OF THE FIGURES

FIG. 1: (a) Schematic of bHGN synthesis. Extinction spectra of (b) sacrificial CoₓB_{y} NP scaffold and (c) resultant HGNs. HGN₁₋₅ were made with equivalent amount of gold and increasing amounts of NaOH, with resultant pH as indicated in Table 1. (d-h) Corresponding SEM and TEM images of HGN₁₋₅ showing surface morphology control. SEM scale bars 100 nm, TEM scale bars 50 nm.

FIG. 2: HRTEM images of HGN₂₋₅ surface protrusions and crystal lattice at two magnifications. Scale bars 5 nm.

FIG. 3: Optical and Structural Characterization of Structures Selected for Heat Generation Comparison. (a) Extinction spectra of HGN_{A-C}. Schematic representation of resultant shells with corresponding SEM images for (b) HGN_{A}: smooth surface with 56±9 nm outer diameter, (c) HGN_{B}: bumpy surface with 70±10 nm outer diameter, (d) HGN_{C}: bumpy surface with 90±10 nm outer diameter. Scale bars 50 nm. HGN_{A-C} have equivalent inner diameter but vary in shell morphology. Synthetic parameters are provided in Table 1.

FIG. 4: Heat generation and cooling curves for HGN_{A-C} at (a) 0.20 OD and (b) 0.50 OD extinction, as measured at 790 nm. (c) Experimental PCE values.

FIG. 5: Representative example of fitting of the cooling curve for HGNC at 0.20 OD extinction (at 790 nm). The temperature decay was fit with Equation 11.

FIG. 6: (a) Schematic to highlight three temperature regions of the cuvette: cuvette walls in contact with HGN solution (Region I), cuvette walls not in contact with HGN solution (Region II), and top of cuvette (Region III). (b) Temperature measurements along the height of the cuvette, reported as the difference between ambient temperature and that of the cuvette at steady state. Data are shown as filled circles and the fit shown as a solid line. Region II was fit with an exponential decay. The integral of the temperature gradient was used to determine the effective mass of the cuvette.

FIG. 7: Extinction of 0.20 OD HGN_{A-C} before and after heat generation experiments to demonstrate photostability.

FIG. 8: Schematic illustration of performing a galvanic exchange reaction at a selected pH to produce HMNs having the pre-selected surface rugosity, where the pH of the galvanic exchange reaction is selected to produce the pre-selected surface rugosity of the HMNs.

### DETAILED DESCRIPTION

Provided are methods of producing hollow metal nanospheres (HMNs) having a pre-selected surface rugosity. The methods include combining in a galvanic exchange reaction at a selected pH: a solution comprising cobalt-based nanoparticle (CoₓB_{y} NP) scaffolds; and a solution comprising a metal, to produce CoₓB_{y} NP core/metal shell structures. The methods further include oxidizing the CoₓB_{y} NP cores of the CoₓB_{y} NP core/metal shell structures to produce HMNs having the pre-selected surface rugosity, where the pH of the galvanic exchange reaction is selected to produce the pre-selected surface rugosity of the HMNs. Also provided are HMNs produced according to the methods, as well as methods of using the HMNs. Compositions and kits that find use, e.g., in practicing the methods of the present disclosure, are also provided.

Before the methods, HMNs, compositions and kits of the present disclosure are described in greater detail, it is to be understood that the methods, HMNs, compositions and kits are not limited to particular embodiments described, as such may, of course, vary. It is also to be understood that the terminology used herein is for the purpose of describing particular embodiments only, and is not intended to be limiting, since the scope of the methods, HMNs, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, HMNs, compositions and kits. The upper and lower limits of these smaller ranges may independently be included in the smaller ranges and are also encompassed within the methods, HMNs, compositions and kits, subject to any specifically excluded limit in the stated range. Where the stated range includes one or both of the limits, ranges excluding either or both of those included limits are also included in the methods, HMNs, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, HMNs, compositions and kits belong. Although any methods, HMNs, compositions and kits similar or equivalent to those described herein can also be used in the practice or testing of the methods, HMNs, compositions and kits, representative illustrative methods, HMNs, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, HMNs, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, HMNs, compositions and kits, which are, for clarity, described in the context of separate embodiments, may also be provided in combination in a single embodiment. Conversely, various features of the methods, HMNs, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, HMNs, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Producing Hollow Metal Nanospheres

As summarized above, the present disclosure provides methods of producing hollow metal nanospheres. The methods include combining in a galvanic exchange reaction at a selected pH: a solution comprising cobalt-based nanoparticle (CoₓB_{y} NP) scaffolds; and a solution comprising a metal, to produce CoₓB_{y} NP core/metal shell structures. The methods further include oxidizing the CoₓB_{y} NP cores of the CoₓB_{y} NP core/metal shell structures to produce HMNs having the pre-selected surface rugosity, where the pH of the galvanic exchange reaction is selected to produce the pre-selected surface rugosity of the HMNs. The amount of metal in the galvanic exchange reaction may also be selected, in combination with the selected pH, to produce the pre-selected surface rugosity of the HMNs. The methods are based in part on the finding that a facile pH modification to previous HMN synthesis methods enables controlled tuning of the surface morphology from smooth to very rugose. Unlike previous approaches, the methods of the present disclosure do not require harsh surfactants, secondary reducing agents, or organic solvents. The resultant rugose HMNs (sometimes referred to herein as “bumpy HMNs” or “bHMNs”, or “bumpy HGNs” or “bHGNs” when the HMNs are hollow gold nanospheres (HGNs)) are highly monodisperse with little variation in protrusion length from particle to particle. In combining the benefits of a hollow core with those of controlled surface morphology, the HMNs produced according to the methods of the present disclosure enable powerful platforms, e.g., for nanotheranostic applications. For example, the HMNs produced according to the methods of the present disclosure exhibit excellent photothermal conversion efficiency (PCE). Further details regarding the methods of making HMNs will now be described.

“Surface rugosity” may refer to a measure of small-scale variations of amplitude in the height of a surface. In one approach for quantifying surface rugosity, surface rugosity may be referred to as “fᵣ”, where

fᵣ=Aᵣ/A_{g},

where Aᵣ is the real (true, actual) surface area and A_{g} is the geometric surface area.

Rugosity may also be quantified as the ratio of the actual circumference of the HMN to the geometric circumference of the HMN, where: the “actual circumference” is the longest line that could be drawn around the perimeter of the HMN (that is, including the bumpy features of the particle); and the “geometric circumference” is the largest circle that fits completely internal to the HMN (that is, no part of the circle would be external to the surface of the HMN). In certain embodiments, when the surface rugosity is quantified as the ratio of the actual circumference to the geometric circumference, the pre-selected surface rugosity of the HMNs is greater than 1 and up to 20, greater than 1 and up to 18, greater than 1 and up to 16, greater than 1 and up to 14, greater than 1 and up to 12, greater than 1 and up to 10, greater than 1 and up to 8, greater than 1 and up to 6, greater than 1 and up to 4, greater than 1 and up to 2, or greater than 1 and up to 1. In some embodiments, when the surface rugosity is quantified as the ratio of the actual circumference to the geometric circumference, the pre-selected surface rugosity of the HMNs is from 1.1 to 5, from 5 to 10, from 10 to 15, or from 15 to 20. In some embodiments, when the surface rugosity is quantified as the ratio of the actual circumference to the geometric circumference, the pre-selected surface rugosity of the HMNs is from 1.1 to 4, from 4 to 8, from 8 to 12, from 12 to 16, or from 16 to 20. In some embodiments, when the surface rugosity is quantified as the ratio of the actual circumference to the geometric circumference, the pre-selected surface rugosity of the HMNs is from 1.1 to 3, from 3 to 6, from 6 to 9, from 9 to 12, from 12 to 15, from 15 to 18, or from 18 to 21. In some embodiments, when the surface rugosity is quantified as the ratio of the actual circumference to the geometric circumference, the pre-selected surface rugosity of the HMNs is from 1.1 to 2, from 2 to 4, from 4 to 6, from 6 to 8, from 8 to 10, from 10 to 12, from 12 to 14, from 14 to 16, from 16 to 18, or from 18 to 20.

By “pre-selected” surface rugosity is meant the practitioner of the subject methods selects a desired surface rugosity of the HMNs prior to production of the HMNs, where the desired surface rugosity of the HMNs is achieved by appropriate selection of the pH of the galvanic exchange reaction, where the selected pH of the galvanic exchange reaction determines the resulting surface rugosity of the HMNs. As demonstrated herein, the pH of the galvanic exchange reaction and the surface rugosity of the HMNs are positively correlated—that is, a higher pH (more basic) galvanic exchange reaction results in a higher degree of surface rugosity, as compared to a lower pH.

According to some embodiments, the pH of the solution comprising the metal is selected to produce the selected pH of the galvanic exchange reaction. In some embodiments, the selected pH of the solution comprising the metal is produced by combining a solution comprising the metal with a basic solution. According to some embodiments, the basic solutions comprises a base comprising a hydroxide group. Non-limiting examples of basic solutions which may be employed when practicing the methods of the present disclosure include sodium hydroxide, potassium hydroxide, calcium hydroxide, ferrous hydroxide, and the like.

As summarized above, the methods of producing the HMNs include a galvanic exchange reaction. As such, the solution comprising the metal comprises a metal which can undergo galvanic exchange with cobalt, e.g., a metal which has a higher reduction potential than cobalt. Details regarding HMN syntheses involving galvanic exchange reactions are provided in International Patent Application Publication Nos. WO/2018/102765 and WO/2018/170304, the disclosures of which are incorporated herein by reference in their entireties for all purposes. In certain embodiments, the galvanic exchange reaction is performed in an anaerobic environment. According to some embodiments, the solution including the metal is deaerated prior to the combining with the solution including the CoₓB_{y} NP scaffolds. By way of example, if the HMNs to be produced are hollow gold nanospheres (HGNs), the solution including the metal may be a deaerated solution including gold, a non-limiting example of which is a deaerated chloroauric acid (HAuCl₄) solution. As used herein, “deaerated” means dissolved air or gas (e.g., oxygen) has been partially or completely removed from a liquid, e.g., a solution. For example, “deaerated” may mean that—as compared to the liquid prior to deaeration—dissolved air or gas in the liquid is reduced by about 50% or more, 60% or more, 70% or more, 80% or more, 90% or more, 95% or more, 98% or more, 99% or more, or by about 100%. Any suitable approaches for deaerating a liquid of interest may be employed. In one non-limiting example, deaeration is performed by bubbling the solution with an inert gas (e.g., nitrogen, argon, helium, or the like) for a suitable period of time, e.g., 1 hour or more. A vacuum gas manifold, such as a Schlenk line, may be used to deaerate a solution of interest.

As summarized above, the methods of producing the HMNs include oxidizing the CoₓB_{y} NP cores of the CoₓB_{y} NP core/metal shell structures to produce HMNs having the pre-selected surface rugosity. In some embodiments, the oxidizing is by oxygenation. In certain aspects, the oxygenation is controlled oxygenation. Controlled oxygenation may include introducing air or pure oxygen into a previously anaerobic environment containing the CoₓB_{y} NP core/metal shell structures produced by galvanic exchange. The rate of introduction may be controlled, e.g., by stirring a solution including the CoₓB_{y} NP core/metal shell structures in an aerobic environment at a selected rate, such as by swirling or using a stir bar and stirring the solution at a selected rpm.

In some embodiments, the methods further include producing the CoₓB_{y} NP scaffolds to be used in the galvanic exchange reaction. Details regarding the production the CoₓB_{y} NP scaffolds for HMN syntheses are provided in International Patent Application Publication Nos. WO/2018/102765 and WO/2018/170304, the disclosures of which are incorporated herein by reference in their entireties for all purposes. The CoₓB_{y} NP scaffolds may be produced using a cobalt salt including Co²⁺ ions. Such a cobalt salt may be any cobalt salt suitable for synthesis of cobalt or cobalt-based nanoparticles, which salt may be selected based on the type of capping agent and/or any other reagents employed for Co-based NP synthesis. In some embodiments, the cobalt salt is an anhydrous cobalt salt. A non-limiting example of an anhydrous cobalt salt that may be employed when practicing the subject methods is CoCl₂. Other suitable cobalt salts include, but are not limited to, CoBr₂, CoI₂, Co(NO₃)₂, Co(acac)₂, Cobalt(II) acetate, etc. In certain embodiments, producing the CoₓB_{y} NP scaffolds comprises nucleating Co²⁺ ions with a sodium borohydride (NaBH₄) solution. According to some embodiments, the NaBH₄ solution has a selected ratio of tetrahydroxyborate (B(OH)₄⁻) to tetrahydroborate (BH₄⁻) to produce CoₓB_{y} NP scaffolds having a pre-selected diameter, where the ratio of B(OH)₄⁻ to BH₄⁻ is positively correlated with the pre-selected diameter of the CoₓB_{y} NP scaffolds.

The HMNs produced according to the methods of the present disclosure may be HMNs that include any metal of interest, including but not limited to, hollow gold nanospheres (HGNs), hollow silver nanospheres (HSNs), etc.

In some embodiments, HMNs (e.g., HGNs) produced in accordance with the methods of the present disclosure have an average diameter of from about 10 to about 200 nm (e.g., from about 20 to about 150 nm), where the average HMN (e.g., HGN) diameter is determined by the diameter of the CoₓB_{y} NPs. For example, the average HMN diameter may be about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 170 nm, 180 nm, 190 nm, or about 200 nm. In some embodiments, the average HMN diameter ranges from about 10 to about 20 nm, 20 to 30 nm, 30 to 40 nm, 40 to 50 nm, 50 to 60 nm, 60 to 70 nm, 70 to 80 nm, 80 to 90 nm, 90 to 100 nm, 100 to 105 nm, 105 to 110 nm, 110 to 115 nm, 115 to 120 nm, 120 to 125 nm, 125 to 130 nm, 130 to 135 nm, 135 to 140 nm, 140 to 145 nm, 145 to 150 nm, 150 to 155 nm, 155 to 160 nm, 160 to 165 nm, 165 to 170 nm, 170 to 175 nm, 175 to 180 nm, 180 to 185 nm, 185 to 190 nm, 190 to 195 nm, or about 195 to about 200 nm. In certain aspects, the average HMN diameter is from about 50 to about 170 nm, from 60 to 160 nm, from 70 to 150 nm, from 80 to 140 nm, from 90 to 130 nm, or from about 100 to about 120 nm. In some embodiments, the average HMN diameter is 10 nm or more, 20 nm or more, 30 nm or more, 40 nm or more, 50 nm or more, 60 nm or more, 70 nm or more, 80 nm or more, 90 nm or more, 100 nm or more, 110 nm or more, 120 nm or more, 130 nm or more, 140 nm or more, 150 nm or more, 160 nm or more, 170 nm or more, 180 nm or more, 190 nm or more, or 200 nm or more. In certain aspects, the average HMN diameter is 200 nm or less, 190 nm or less, 180 nm or less, 170 nm or less, 160 nm or less, 150 nm or less, 140 nm or less, 130 nm or less, 120 nm or less, 110 nm or less, 100 nm or less, 90 nm or less, 80 nm or less, 70 nm or less, 60 nm or less, 50 nm or less, 40 nm or less, 30 nm or less, 20 nm or less, or 10 nm or less. In some embodiments, the average HMN (e.g., HGN) diameter is from about 45 nm to about 55 nm, e.g., from about 46 nm to about 54 nm, from about 47 nm to about 53 nm, from about 48 to about 52 nm, or from about 49 to about 51 nm, e.g., about 50 nm.

As will be appreciated, the diameters of individual HMNs produced according to subject methods will vary around the average diameter. In some embodiments, the diameters of the HMNs produced will vary around the average diameter (e.g., any of the average diameters provided in the preceding paragraph) by 20% or less, 17.5% or less, 15% or less, 12.5% or less, 10% or less, 7.5% or less, 5% or less, 2.5% or less, 2.0% or less, 1.5% or less, or 1% or less.

The monodispersity of HMNs produced according to the present methods may be expressed in terms of relative standard deviation (RSD). In some embodiments, the HMNs produced exhibit an RSD of 5% or less, 4.5% or less, 4% or less, 3.5% or less, 3% or less, 2.5% or less, 2% or less, 1.5% or less, or 1% or less.

HMNs (e.g., HGNs) produced according to the methods of the present disclosure may exhibit optical properties, photothermal properties, and/or the like determined by their pre-selected surface rugosity, average diameter, aspect ratio, and/or the like. In some embodiments, the HMNs (e.g., HGNs) exhibit a surface plasmon resonance (SPR) absorption with a maximum peak position of from about 565 to about 1300 nm. In certain aspects, the produced HMNs exhibit a SPR absorption with a maximum peak position in the visible range, e.g., from about 400 to about 700 nm. In some embodiments, the produced HMNs (e.g., HGNs) exhibit an SPR absorption with a maximum peak position in the infrared range (which is 700 nm to 1 mm), e.g., from about 700 nm to about 1 μm. In certain aspects, the produced HMNs (e.g., HGNs) exhibit an SPR absorption with a maximum peak position in the near-infrared (near-IR) range, e.g., from about 700 nm to about 2500 nm. In some embodiments, the produced HMNs (e.g., HGNs) exhibit an SPR absorption with a maximum peak position of from about 400 to about 1200 nm (e.g., from about 565 to about 850 nm), such as from 420 to 1180 nm, from 440 to 1160 nm, from 460 to 1140 nm, from 480 to 1120 nm, from 500 to 1100 nm, from 520 to 1080 nm, from 540 to 1060 nm, from 560 to 1040 nm, from 580 to 1020 nm, from 600 to 1000 nm, from 620 to 980 nm, from 640 to 960 nm, from 660 to 940 nm, from 680 to 920 nm, from 700 to 900 nm, from 720 to 880 nm, from 740 to 860 nm, from 760 to 840 nm, from 780 to 820 nm, from 785 to 815 nm, from 790 to 810 nm, or from about 795 to about 805 nm, e.g., about 800 nm.

In some embodiments, the methods of producing HMNs may further include attaching a moiety (e.g., a targeting moiety) to the surface of the HMNs. In certain aspects, a targeting moiety selected from an antibody, a ligand, an aptamer, a nucleic acid, and a small molecule, is attached to the surface of the HMNs. By “targeting moiety” is meant a moiety that directly or indirectly binds to a target. Targets of interest include analytes (e.g., proteins, nucleic acids, small molecules, or the like), cells (e.g., cells in an in vitro or in vivo environment), and the like.

In certain aspects, the HMNs include a targeting moiety (e.g., an antibody, cell surface receptor ligand, or the like) that binds to a molecule on the surface of a target cell in vitro or in vivo. Such HMNs find use in research, diagnostic, and/or therapeutic applications. In some embodiments, the target cell is a cancer cell. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In some embodiments, the HMNs include a targeting moiety (e.g., an antibody, cell surface receptor ligand, or the like) that binds to a tumor-associated or tumor-specific cell surface molecule, e.g., cell surface receptor, membrane protease, and the like. By “tumor-associated cell surface molecule” is meant a cell surface molecule expressed on malignant cells with limited expression on cells of normal tissues, or a cell surface molecule expressed at much higher density on malignant versus normal cells.

Any tumor-associated cell surface molecule or tumor-specific cell surface molecule may be targeted by the HMNs of the present disclosure. In certain aspects, the target on the cancer cell surface to which the targeting moiety of the HMNs binds is EGFR, HER2, CD19, CD22, CD30, CD33, CD56, CD66/CEACAM5, CD70, CD74, CD79b, CD138, Nectin-4, Mesothelin, Transmembrane glycoprotein NMB (GPNMB), Prostate-Specific Membrane Antigen (PSMA), SLC44A4, CA6, CA-IX, avβ1 integrin, avβ3 integrin, avβ5 integrin, avβ6 integrin, a5β1 integrin, neuropilin-1 (NRP1), matriptase, or any other tumor-associated or tumor-specific cell surface molecule of interest.

A variety of suitable approaches exist for attaching a targeting moiety to HMNs. In one non-limiting example, thiol-based surface functionalization of the HMNs may be employed. For example, bifunctional SH-PEG-COOH linkers have been employed to conjugate antibodies to HMNs, the details of which may be found, e.g., in Liu et al. (2015) Nanoscale Res. Lett. 10:218. Briefly, the SH-PEG-COOH linker may be reacted with the HMNs, followed by addition of N-(3-dimethylaminopropyl)-N-ethylcarbodiimidehydrochloride (EDC) and N-hydroxy succinimide (NHS) to activate the carboxyl terminal of PEG, followed by combining the PEGylated HMNs with the antibody of interest. In some embodiments, an orthopyridyl disulfides-poly(ethylene) glycol-succinimidyl valerate (OPSS-PEG-SVA) linker is used to attach a targeting moiety to HMNs.

Also provided are HMNs (e.g., HGNs) produced according to any of the methods of the present disclosure. The HMNs may be present in a container, such as a vial, tube, plate (e.g., 96-well or other plate), flask, or the like. In some embodiments, the HMNs are present in a liquid medium, e.g., water or other suitable liquid storage medium. In certain aspects, the HMNs are present in a lyophilized form.

The present disclosure also provides methods of using the produced HMNs (e.g., HGNs) in a variety of applications. Non-limiting examples of such applications include surface-enhanced Raman scattering (SERS), photothermal therapy (PTT), plasmonic enhanced photoelectric conversion, chemical catalysis and biosensors.

In one example, HMNs (e.g., HGNs) produced according to the methods of the present disclosure are used for photothermal therapy (PTT). PTT involves embedding nanoparticles within tumors, which nanoparticles generate heat in response to exogenously applied laser light, thereby killing tumor cells in the vicinity of the nanoparticles. The preferred mediators of PTT are gold-based nanoparticles because they offer: (1) simple gold-thiol bioconjugation chemistry for the attachment of desired targeting molecules; (2) biocompatibility, (3) efficient light-to-heat conversion; (4) small diameters that enable tumor penetration upon systemic delivery, and (5) the ability to be tuned to absorb near-infrared light, which penetrates tissue more deeply than other wavelengths of light. PTT may be used in combination with other therapies, such as chemotherapy, gene regulation, and immunotherapy, for enhanced anti-tumor effects. Details regarding PTT approaches that may be practiced employing HMNs produced according to methods of the present disclosure may be found, e.g., in Riley R. S. & Day, E. S. (2017) Wiley Interdiscip. Rev. Nanomed. Nanobiotechnol. 2017 9(4); Melancon et al. (2008) Mol. Cancer Ther. 7:1730; and Lu et al. (2009) Clin. Cancer Res. 15:876.

Accordingly, provided are methods that include administering HMNs (e.g., HGNs) produced according to the methods of the present disclosure to an individual in need thereof. In some embodiments, the individual in need thereof is in need of photothermal therapy (PTT), e.g., an individual having cancer. In certain aspects, the HMNs (e.g., HGNs) include a targeting moiety that binds to a molecule on the surface of a target cell (e.g., a cancer cell) of the individual.

A variety of subjects are treatable according to the subject methods. Generally, such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the subject is a human.

In certain embodiments, the subject has a cancer characterized by the presence of a solid tumor, a semi-solid tumor, a primary tumor, a metastatic tumor, or the like. In some embodiments, the subject has a cancer selected from breast cancer, melanoma, lung cancer, colorectal cancer, prostate cancer, glioma, glioblastoma, bladder cancer, endometrial cancer, kidney cancer, leukemia (e.g., acute myeloid leukemia (AML)), liver cancer (e.g., hepatocellular carcinoma (HCC), such as primary or recurrent HCC), non-Hodgkin lymphoma, pancreatic cancer, thyroid cancer, any combinations thereof, and any sub-types thereof.

According to some embodiments, the methods of the present disclosure (e.g., phototherapy-based methods) are effective in treating the tumor of the subject. By “treat”, “treating” or “treatment” is meant at least an amelioration of the symptoms associated with a medical condition of the subject (e.g., cell proliferative disorder, e.g., cancer) of the subject, where amelioration is used in a broad sense to refer to at least a reduction in the magnitude of a parameter, e.g. symptom, associated with the medical condition being treated. As such, treatment also includes situations where the medical condition, or at least symptoms associated therewith, are completely inhibited, e.g., prevented from happening, or stopped, e.g., terminated, such that the subject no longer suffers from the medical condition, or at least the symptoms that characterize the medical condition.

A pharmaceutical composition comprising the HMNs of the present disclosure is administered to the subject in an effective amount. By “effective amount” is meant a dosage sufficient to produce a desired result, e.g., an amount sufficient to effect beneficial or desired photothermal therapeutic (PTT) results, such as a reduction in a symptom of cancer, as compared to a control. In some embodiments, an effective amount is sufficient to slow the growth of a tumor, reduce the size of a tumor, and/or the like. An effective amount may be administered in one or more administrations.

The pharmaceutical composition may be administered to the subject using any available method and route suitable for nanosphere delivery, including in vivo and ex vivo methods, as well as systemic and localized routes of administration. Conventional and pharmaceutically acceptable routes of administration include intranasal, intramuscular, intra-tracheal, subcutaneous, intradermal, topical application, ocular, intravenous, intra-arterial, nasal, oral, and other enteral and parenteral routes of administration. In some embodiments, the administering is by parenteral administration. Routes of administration may be combined, if desired, or adjusted depending upon the particular nanospheres and/or the desired effect. The pharmaceutical compositions may be administered in a single dose or in multiple doses. In some embodiments, the pharmaceutical composition is administered intravenously. In some embodiments, the pharmaceutical composition is administered by injection, e.g., for systemic delivery (e.g., intravenous infusion) or to a local site.

### Compositions

Also provided are compositions that include the HMNs (e.g., HGNs) produced according to the methods of the present disclosure. The compositions may include any of the HMNs described herein. In certain aspects, the compositions include the HMNs present in a liquid medium. The liquid medium may be an aqueous liquid medium, such as water, a buffered solution, and the like. One or more additives such as a salt (e.g., NaCl, MgCl2, KCl, MgSO4), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), glycerol, a chelating agent, and the like may be present in such compositions.

Pharmaceutical compositions are also provided. The pharmaceutical compositions include any of the HMNs (e.g., HGNs) of the present disclosure, and a pharmaceutically acceptable carrier. The pharmaceutical compositions generally include a therapeutically effective amount of the HMNs, e.g., for use in photothermal therapy. By “therapeutically effective amount” is meant a dosage sufficient to produce a desired result, e.g., an amount sufficient to effect beneficial or desired therapeutic (including preventative) results, such as a reduction in a symptom of a disease or disorder (e.g., a cell proliferative disorder such as cancer), as compared to a control. An effective amount can be administered in one or more administrations.

The HMNs of the present disclosure can be incorporated into a variety of formulations for therapeutic administration, e.g., oral, parenteral, or other routes of administration. More particularly, the HMNs can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the HMNs suitable for administration to a patient (e.g., suitable for human administration) are generally sterile and may further be free of detectable pyrogens or other contaminants contraindicated for administration to a patient according to a selected route of administration.

The HMNs can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the HMNs may be prepared by mixing the HMNs having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

### Kits

As summarized above, the present disclosure provides kits. In some embodiments, provided are kits that include hollow metal nanospheres (HMNs, such as HGNs) produced according to the methods of the present disclosure, or a composition (e.g., pharmaceutical composition) including such HMNs. Such kits may include instructions for employing the HMNs in a variety of research, diagnostic and/or therapeutic applications. In certain aspects, the kits include instructions for using the HMNs to detect an analyte in vitro (e.g., biosensing, such as in vitro analyte detection, or the like) or in vivo (e.g., in vivo imaging, such as in vivo tumor imaging, or the like). Alternatively, or additionally, the kits may include instructions for administering the HMNs to an individual in need thereof, e.g., an individual in need of photothermal therapy (PTT), such as an individual having cancer. Kits that include HMNs for therapeutic applications may include the HMNs present in one or more (e.g., two or more) unit dosages.

Kits that include HMNs may further include one or more reagents and accompanying instructions for functionalizing the surface of the HGNs, e.g., by attaching a linker and/or moiety (e.g., a targeting moiety such as an antibody, or the like) to the surface of the HMNs. In one example, the kits include a thiol-based surface functionalization reagent, e.g., a bifunctional thiol-based linker, such as an SH-PEG-COOH linker. In some embodiments, the targeting moiety is one that binds to a molecule on the surface of a target cell (e.g., a cancer cell) in vitro or within an individual.

In some embodiments, provided are kits that include any of the HMNs, compositions, or pharmaceutical compositions of the present disclosure, and instructions for using the HMNs to detect an analyte in vitro or in vivo. In certain aspects, provided are kits that include any of the HMNs, compositions, or pharmaceutical compositions of the present disclosure, and instructions for administering the HMNs to an individual in need thereof.

Components of the kits may be present in separate containers, or multiple components may be present in a single container. For example, in a kit that includes one or more reagents for functionalizing the surface of the HMNs, two or more of such reagents may be provided in the same tube, or may be provided in different tubes.

In addition to the above-mentioned components, and as described above, a subject kit may further include instructions for using the components of the kit, e.g., to practice the methods of the present disclosure. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Notwithstanding the appended claims, the present disclosure is also defined by the following embodiments.
1. A method of producing hollow metal nanospheres (HMNs) having a pre-selected surface rugosity, comprising:
combining in a galvanic exchange reaction at a selected pH:
   a solution comprising cobalt-based nanoparticle (CoₓB_{y} NP) scaffolds; and
   a solution comprising a metal,
   to produce CoₓB_{y} NP core/metal shell structures; and
oxidizing the CoₓB_{y} NP cores of the CoₓB_{y} NP core/metal shell structures to produce HMNs having the pre-selected surface rugosity,
wherein the pH of the galvanic exchange reaction is selected to produce the pre-selected surface rugosity of the HMNs.
   2. The method according to embodiment 1, wherein the pH of the solution comprising the metal is selected to produce the selected pH of the galvanic exchange reaction.
   3. The method according to embodiment 2, wherein the selected pH of the solution comprising the metal is produced by combining a solution comprising the metal with a basic solution.
   4. The method according to embodiment 3, wherein the basic solution is sodium hydroxide.
   5. The method according to any one of embodiments 1 to 4, wherein the galvanic exchange reaction is performed in an anaerobic environment.
   6. The method according to any one of embodiments 1 to 5, wherein the solution comprising the metal is deaerated prior to the combining with the solution comprising the CoₓB_{y} NP scaffolds.
   7. The method according to any one of embodiments 1 to 6, wherein the oxidizing is by oxygenation.
   8. The method according to embodiment 7, wherein the oxygenation is controlled oxygenation.
   9. The method according to any one of embodiments 1 to 8, further comprising producing the CoₓB_{y} NP scaffolds.
   10. The method according to embodiment 9, wherein producing the CoₓB_{y} NP scaffolds comprises nucleating Co²⁺ ions with a sodium borohydride (NaBH₄) solution.
   11. The method according to embodiment 10, wherein the NaBH₄ solution has a selected ratio of tetrahydroxyborate (B(OH)₄⁻) to tetrahydroborate (BH₄⁻) to produce CoₓB_{y} NP scaffolds having a pre-selected diameter, wherein the ratio of B(OH)₄⁻ to BH₄⁻ is positively correlated with the pre-selected diameter of the CoₓB_{y} NP scaffolds.
   12. The method according to any one of embodiments 1 to 11, wherein the HMNs are hollow gold nanospheres (HGNs).
   13. The method according to embodiment 12, wherein the solution comprising the metal is chloroauric acid (HAuCl₄).
   14. The method according to embodiment 13, wherein the pH of the HAuCl₄ is selected to produce the selected pH of the galvanic exchange reaction.
   15. The method according to embodiment 14, wherein the selected pH of the HAuCl₄ is produced by combining HAuCl₄ with a basic solution.
   16. The method according to embodiment 15, wherein the basic solution is sodium hydroxide.
   17. The method according to any one of embodiments 1 to 16, wherein the HMNs exhibit a surface plasmon resonance (SPR) absorption with a maximum peak position of from about 565 to about 1300 nm.
   18. The method according to any one of embodiments 1 to 17, further comprising, subsequent to producing the HMNs, attaching a targeting moiety to the surface thereof.
   19. The method according to embodiment 18, wherein the targeting moiety is selected from the group consisting of: an antibody, a ligand, an aptamer, a nucleic acid, and a small molecule.
   20. The method according to embodiment 18 or embodiment 19, wherein the targeting moiety binds to a molecule on the surface of a target cell.
   21. The method according to embodiment 20, wherein the target cell is a cancer cell.
   22. Hollow metal nanospheres (HMNs) produced according to the methods of any one of embodiments 1 to 21.
   23. A composition comprising the HMNs of embodiment 22.
   24. A pharmaceutical composition, comprising:
the HMNs of embodiment 22; and
a pharmaceutically acceptable carrier.
   25. A kit, comprising:
the HMNs of embodiment 22, the composition of embodiment 23, or the pharmaceutical composition of embodiment 24; and
instructions for using the HMNs to detect an analyte in vitro or in vivo.
   26. A kit, comprising:
the HMNs of embodiment 22, the composition of embodiment 23, or the pharmaceutical composition of embodiment 24; and
instructions for administering the HMNs to an individual in need thereof.
   27. A method comprising administering to an individual in need thereof the HMNs of embodiment 22, the composition of embodiment 23, or the pharmaceutical composition of embodiment 24.
   28. The method according to embodiment 27, wherein the individual in need thereof is in need of photothermal therapy (PTT).
   29. The method according to embodiment 27 or embodiment 28, wherein the individual has a cell proliferative disorder.
   30. The method according to embodiment 29, wherein the cell proliferative disorder is cancer.
   31. The method according to embodiment 30, wherein the HMNs are targeted to cancer cells present in the individual via a targeting moiety on the surface of the HMNs.

The following examples are offered by way of illustration and not by way of limitation.

### EXPERIMENTAL

### Example 1—Optical and Structural Properties of CoₓB_{y} Scaffolds and Resultant bHGNs

HGNs were synthesized through galvanic exchange with sacrificial cobalt-based NP templates. The synthesis involves three main steps: 1) CoₓB_{y} NPs were prepared by a reaction between aqueous cobalt chloride and sodium borohydride in the presence of a citrate capping ligand, 2) galvanic exchange was carried out between the CoₓB_{y} NP scaffold and chloroauric acid, and 3) the residual CoₓB_{y} NP cores were oxidized into solution by exposure to environmental oxygen, leaving behind solvent-filled shells of gold.

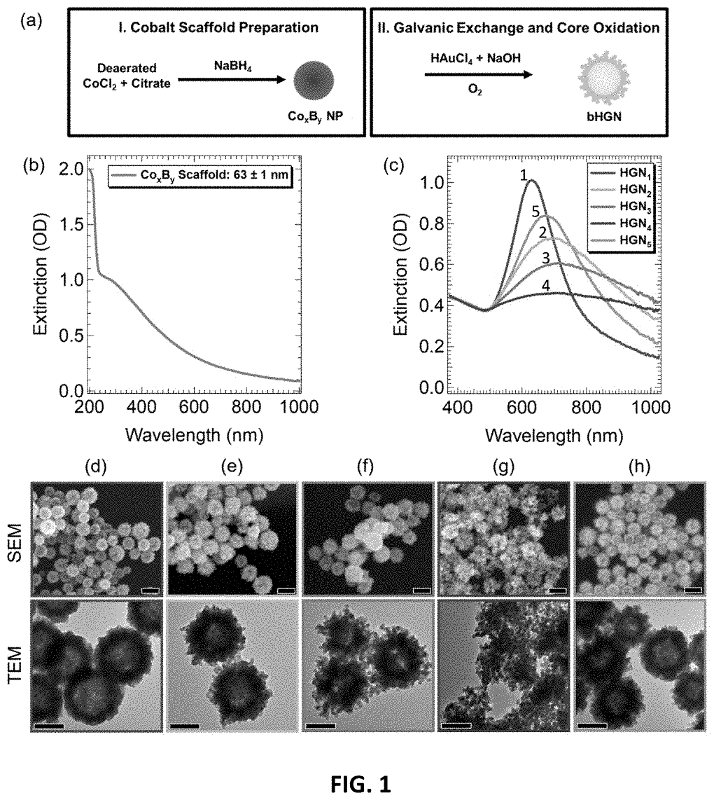

In this work, CoₓB_{y} NP scaffolds were prepared with BH₄⁻:Co²⁺ and B(OH)₄⁻:BH₄⁻ molar ratios of 3.00 and 1.67, respectively. It was previously demonstrated that BH₄⁻:Co²⁺ and B(OH)₄⁻:BH₄⁻ ratios may be used to control scaffold size and monodispersity. The resultant scaffolds were characterized using dynamic light scattering (DLS) and UV-Vis spectroscopy. The hydrodynamic diameter of the scaffolds was 63±1 nm and the extinction spectrum followed normal exponential decay with a feature appearing ˜280 nm, as expected for this size (FIG. 1, panel b).

In converting the scaffolds to bHGNs, an aerobic protocol was used for galvanic exchange as it promotes patchwork deposition of gold and can therefore facilitate the formation of rugose structures. In this protocol, Steps 2 and 3 occur simultaneously, as shown in the schematic provided in FIG. 1, panel a. Additionally, a large amount of HAuCl₄ was used to ensure enough material was available for the formation of highly rugose shells. Since pH-mediated HAuClₓOH₄₋ₓ speciation has previously been implicated in morphology control of solid gold structures, the addition of NaOH during galvanic exchange was assessed.

It was found that NaOH may be used during galvanic exchange to controllably tune the surface features of HGNs. In absence of NaOH, the resultant shells were thick and nodular due to the large amount of Au³⁺ provided, as seen in FIG. 1, panel d (HGN₁). When 10 μmol NaOH was added to the gold solution before galvanic exchange, the pH of the gold solution increased from 3.33 to 4.16 and the nodules of the resultant shells were elongated into protrusions, as shown in FIG. 1, panel e (HGN₂). When 20 μmol NaOH was used, this effect was exaggerated; the pH of the gold solution increased to 10.5 and the resultant protrusions became even more elongated, creating highly rugose surfaces (HGN₃). Because these structures were synthesized from the same sacrificial scaffold, their hollow cores can be expected to have similar dimensions. The creation of surface features, however, increased the outer diameter from 70±10 nm for HGN₁ to 90±10 nm for HGN₃. The low relative standard deviation for these structures indicates a high degree of uniformity. Although the surfaces are highly rugose, and although the bumps seem somewhat randomly distributed, all protrusions reach similar lengths from their common core and are thus well-controlled. When 30 μmol NaOH is used, resulting in a pH of 11.0, the resultant HGNs are so bumpy that they lose structural integrity and comprise a mixture of gold fragments loosely held together in spherical superstructures (FIG. 1, panel g, HGN₄). In FIG. 1, SEM is provided to highlight the surface features of each shell and TEM is provided to clearly demonstrate the presence of the hollow core.

The change in surface morphology of HGNs in turn affects their optical properties. For HGN₁, with no addition of NaOH, the SPR was strong and symmetric around 630 nm. As the structure became bumpier, the SPR broadened and red-shifted. When the shells lost structural integrity, the resultant extinction was lower and broader, barely resembling an SPR. For all samples, synthetic parameters, structural dimensions, and peak SPR wavelength are tabulated in Table 1. The reported pH measurements were taken on the gold solutions 60 seconds after NaOH addition and immediately before initiating cobalt transfer.

TABLE 1
Synthetic Parameters for Galvanic Exchange and Resultant
Structural Properties for HGN₁₋₅ and HGN_{A-C}

| | | | | | | Inner | Outer |
|---|---|---|---|---|---|---|---|
| | HAuCl₄ | NaOH | Time | | Surface | Diameter | Diameter |
| HGN | (μmol) | (μmol) | (min) | pH | Morphology | (nm) | (nm) |
| 1 | 4.0 | 0 | — | 3.33 | Nodular | 46 ± 8 | 70 ± 10 |
| 2 | 4.0 | 10. | 1 | 4.16 | Bumpy | 46 ± 8 | 80 ± 10 |
| 3 | 4.0 | 20. | 1 | 10.5 | Bumpy | 46 ± 8 | 90 ± 10 |
| 4 | 4.0 | 30. | 1 | 11.0 | Fragmented | — | — |
| 5 | 4.0 | 30. | 60 | 7.60 | Bumpy | 46 ± 8 | 70 ± 10 |
| A | 0.40 | 0 | — | — | Smooth | 46 ± 8 | 56 ± 9 |
| B | 0.80 | 4.0 | 1 | 9.45 | Bumpy | 46 ± 8 | 70 ± 10 |
| C | 4.0 | 20. | 1 | 10.5 | Bumpy | 46 ± 8 | 90 ± 10 |

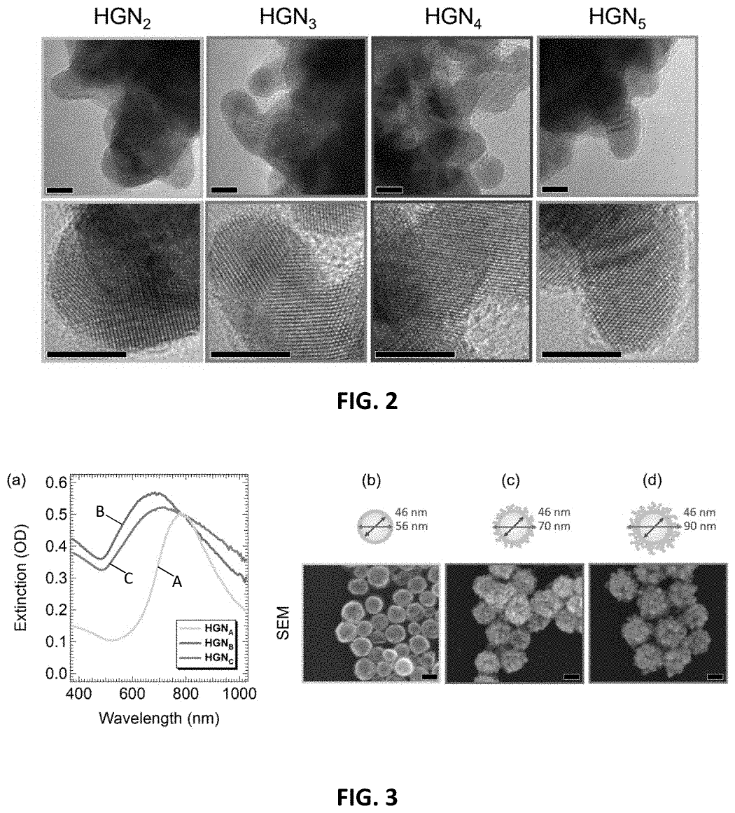

High resolution TEM (HRTEM) of the surface protrusions at two magnifications are provided in FIG. 2 for HGN₂₋₅. The bumpy features are crystalline with large grain sizes and a low number of grain boundaries per protrusion. The overall shape of each protrusion is much rounder than those of the classic nanostar or nanourchin. The protrusions seem to bend at the tips in random directions, which is also in contrast to the usual straight and radial nature of spiky or urchin-like structures and so likely arise from a different mechanism of formation.

### Example 2—Mechanism of Surface Structuring

To investigate the mechanism behind bHGN formation, 30 μmol NaOH was allowed to equilibrate with 4.0 μmol HAuCl₄ for 1 hour before galvanic exchange. During this time, the pH dropped from 11.0 to 7.60. The resultant HGNs are shown in FIG. 1, panel h. Interestingly, the resultant HGNs were not destabilized into gold fragments like those in FIG. 1, panel g. Instead, they were bumpy shells more closely resembling those of FIG. 1, panels d,e in both structure and extinction. The SPR was centered at 675 nm (HGN₅) and far narrower than the 30 μmol NaOH, pH 11.0 sample (HGN₄). This result indicates that Na⁺ is not responsible for the change in morphology; the same amount of Na⁺ is present in both 30 μmol NaOH samples. Furthermore, the additional Na⁺ from NaOH is small in comparison to the amount already present in solution due to the sodium citrate capping agent and sodium borohydride-based nucleation and growth agents. Thus, the morphology change must be due to the addition of OH⁻.

The decrease in pH while OH⁻ is allowed to equilibrate with the gold salt solution is due to OH⁻ ligands replacing the CI⁻ ligands of HAuCl₄. Speciation of HAuCl₄ is not likely to be the cause for the morphology change in the present system. When the NaOH was initially added, there was a high concentration of free OH⁻ in solution (and thus a high pH). As the solution equilibrated over the course of an hour, the pH steadily dropped, indicative of OH⁻ displacement of the Cl⁻ ligands and therefore less free OH⁻ in solution. Because the resultant HGNs were less bumpy when displacement occurred and pH dropped (FIG. 1, panel h vs. FIG. 1, panel g), we conclude that bump formation is not due to HAuClₓOH₄₋ₓ speciation or the associated change in reactivity. Instead, the surface morphology must be mainly controlled by the presence of free OH⁻ in solution.

Without being bound by theory, it is proposed that with high levels of free OH⁻, liberated Co²⁺ may react with OH⁻ to form a hydroxide layer around the scaffold NP, limiting further access by Au³⁺ and promoting the growth of bumpy surface features. At higher levels of free OH⁻, the layer becomes more complete, only allowing small areas of gold reduction and resulting in the loosely spherical collection of gold fragments once the core is oxidized.

It should be noted that the method reported herein enables tunable surface features and tunable SPR. The amount of gold can be increased or decreased for relatively thicker or thinner shells and the amount of OH⁻ added (and resultant pH) can be used to select the degree of resultant surface protrusions. The method is also generally applicable. It can be used to form hollow rugose structures of other metals as long as the reduction potential is favorable for galvanic exchange with cobalt.

### Example 3—Select Structures for Photothermal Comparison

With an understanding of how to control surface morphology, three structures were synthesized for a systematic investigation into the effect of surface morphology on heat generation. HGN_{A-C} were made from the same scaffold using different volumes of HAuCl₄ and NaOH. As such, they have equivalent inner diameters but vary in shell morphology and thickness. Extinction and structural schematics of each are provided in FIG. 3. HGN_{A} has a smooth shell and TEM reveals an inner diameter of 46±8 nm and an outer diameter of 56±9 nm (FIG. 3, panel b). HGN_{B} has a bumpy shell with outer diameter of 80±10 nm (FIG. 3, panel c). HGN_{C} has a thicker, bumpier shell with an outer diameter of 90±10 nm (FIG. 3, panel d). HGN_{C} is equivalent to HGN₃ above.

### Example 4—Heat Generation and Calculation of Photothermal Conversion Efficiency

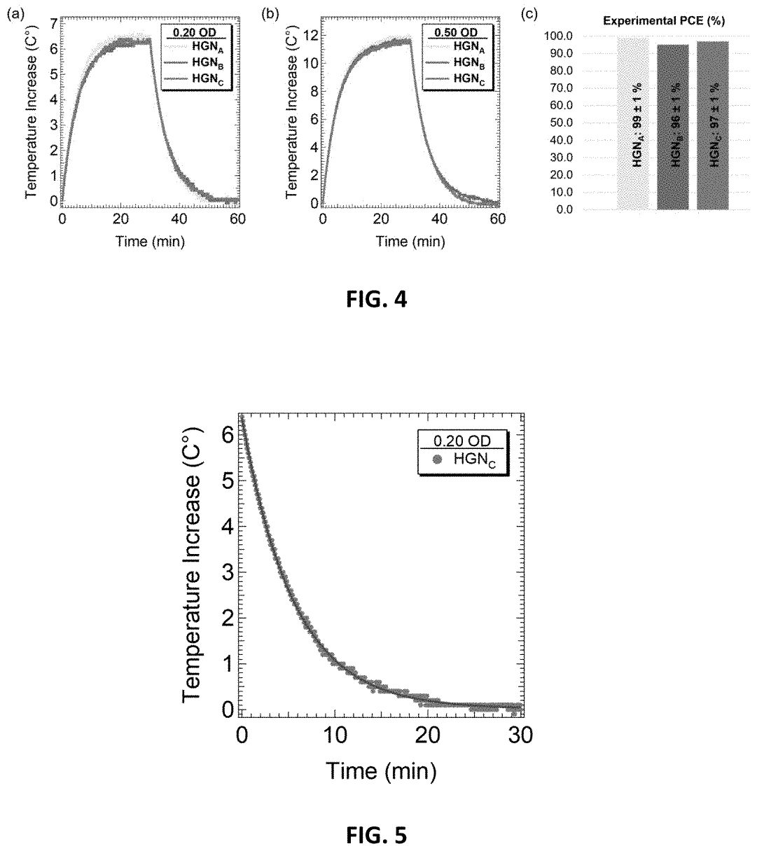

To assess the effect of surface morphology on the ability of the particles to convert light to heat, the change in temperature was recorded for 1.0 mL solutions of HGN samples illuminated by a 790 nm CW laser with 7 mm spot size and 1.0 Wcm⁻² power density. After 30 minutes of irradiation, the laser was blocked and the samples were allowed to cool for 30 minutes. Two particle concentrations were assessed for each sample, corresponding to 0.20 OD and 0.50 OD extinction at 790 nm. Resultant heating and cooling curves are displayed in FIG. 4, panels a,b. Extinction measurements were taken before and after heat generation to confirm photostability during measurement.

Given equivalent optical extinction, HGN_{A-C} resulted in essentially equivalent temperature increase, regardless of surface morphology. The 0.20 OD samples generated 6.0-6.5 C.° and the 0.50 OD samples generated 11.5-12.0 C°. To further characterize the photophysical performance of smooth and bHGN, PCE was calculated. A detailed description of related equations and calculations are provided in Example 5. Briefly, PCE may be calculated using Equation 1

$$η = \frac{B Δ T + C Δ {T}^{2} - I ξ}{I ( 1 - ξ ) ( 1 - {10}^{- E} λ )} \qquad Equation 1$$

where η is PCE, B and C are Taylor series coefficients related to energy dissipation, ΔT is the change in temperature of the solution, I is the reflection-corrected incident laser power, ξ is the fraction of laser energy absorbed by the cuvette and water solvent, and E_{λ} is the extinction at the illumination wavelength. Conceptually, this calculation compares the amount of heat energy generated by the HGNs (numerator) to the amount of incident laser energy extinct by the HGNs (denominator).

B and C may be determined by temporal analysis of the cooling curves when laser illumination is ceased. In this work, B and C were determined to be 2.00×10⁻² WK⁻¹ and 2.25×10⁻⁵ WK⁻², respectively. For ease of comparison with published studies reporting in minute timescales, this equates to 1.20 Jmin⁻¹K⁻¹ and 1.35×10⁻³ Jmin⁻¹K⁻², respectively. C being relatively negligible supports the truncation of the Taylor series after two terms. The fit equation and associated details can be found in Example 5.

Both I and ξ were determined by repeating the photothermal experiment in absence of HGNs. The incident laser power on the system was 0.385 W. After insertion of a cuvette with water solvent, the transmitted power decreased to 0.345 W. The lost 40 mW was extinct by the water and cuvette alone and thus not available to HGNs. The steady state temperature achieved upon water illumination is usually used to determine ξ. For the current work, however, illumination of water produced no measurable temperature increase. Considering 0.3 C.° as our measurable limit, we conclude that at most 6 mW of the incident light contributed to heat generation, while the remaining 34 mW was extinct through non-absorptive means (like reflection). Thus, 6 mW was taken as the Iξ term and 0.351 W was used as the reflection-corrected incident laser power.

Using Equation 1, average PCE values for HGN_{A-C} were calculated to be 99%, 96%, and 97%, respectively. These values are shown in the column chart in FIG. 4, panel c. For comparison, a comprehensive collection of Au nanostructures with previously reported PCE is displayed in Table 2.

TABLE 2
Comparison of PCE for Gold Nanostructures. The SPR column represents
the reported λₘₐₓ. If no λₘₐₓ was reported, NIR-I denotes
SPR occurring in the first NIR window (~650-950 nm) and NIR-II
denotes SPR occurring in the second NIR window (~950-1350 nm).

| Gold | PCE | SPR | λ_{exc} | |
|---|---|---|---|---|
| Nanostructure | (%) | (nm) | (nm) | Reference |
| Hexapod | 29.6 | 805-810 | 808 | 61 |
| Biodegradable | 37 | Broad NIR-I | 808 | 62 |
| Nanovesicle | | | | |
| Nanocup | 38.5 | Broad NIR-I | 808 | 63 |
| Nanoshell | 39 | 800 | 810 | 64 |
| Nanospike | 50.3 | 670 | 808 | 65 |
| Branched | 56 | 840 | 808 | 66 |
| Nanoparticle | | | | |
| Branched | 61 | 740 | 808 | 36 |
| Nanostructure | | | | |
| Nanomatryoshka | 63 | 800 | 810 | 64 |
| Nanocage | 63.6 | 805-810 | 808 | 61 |
| Bellflower | 74 | Broad NIR-I | 808 | 67 |
| Branched | 75.5 | Broad NIR-I | 808 | 46 |
| Nanoporous | | | | |
| Nanoshell | | | | |
| Spiky | 78.8 | Broad NIR-II | 980 | 68 |
| Nanoparticle | | | | |
| Nanobipyramid | 50-90 | 809 | 809 | 60 |
| Nanorod | 22.1-95 | 805-810, 809 | 808, 809 | 60, 61 |
| bHGN | 96-97 | Broad NIR-I | 790 | This Study |
| Smooth HGN | 99 | 790 | 790 | This Study |

The smooth and bumpy HGNs assessed in this study represent the highest reported PCE for Au nanostructures to date. Comparison to the PCE of other common photothermal nanomaterials like metal sulfides and related heterostructures is provided in Table 4. Although an increase in diameter would be expected to correspond with an increase in the scattering component of extinction (and thereby a decrease in the absorption component and resultant PCE), this was not seen this experimentally. The PCE values for HGN_{A} and HGN_{C} are not appreciably different even though the particle diameter increases from 56±9 nm to 90±10 nm. It may be the case that the HGN_{C} does indeed scatter more incident light, but is able to reabsorb the scattered light very efficiently, limiting the effect of increasing size on PCE. Reabsorption has been suggested as an explanation for similar observations in other nanoparticle systems, such as nanobipyramids.

It is clear that HGNs are highly efficient light-to-heat converters and the formation of bumps on the HGN surface does not detract from their photothermal conversion performance in solution, at least for this size regime. The HGN structure is highly tunable; diameters ranging from under 20 nm to over 120 nm have been reported and the absorption maximum has also been shifted into the second NIR region. To shift far into the second NIR region, the HGN may become very large in diameter or very thin in shell. In the latter case, the shell may become so thin that it becomes holey or cage-like. It should be noted that the PCE of nanorods, nanobipyramids, and solid nanostars has been shown to be dimension-dependent with reported PCE values range from 22.1-95% for nanorods and 50-90% for nanobipyramids. Further investigation is needed to reveal how diameter, shell thickness, and aspect ratio affect PCE for HGNs.

Interestingly, PCE may also be understood through analysis of transmitted light. For the 0.20 OD samples, the denominator for Equation 1 reveals that 127 mW of incident light was made extinct by the HGNs. As described above, of the 385 mW incident light, 40 mW was extinct by the cuvette and solvent system alone. For the 0.2 OD samples, 220 mW of incident light was transmitted, indicating that 125 mW was made extinct by the HGNs. This number is in good agreement with the 127 mW determined through Equation 1. Likewise, for the 0.50 OD measurements, transmitted power analysis reveals 235 mW of extinct light which is again in very good agreement with the Equation 1 denominator of 236 mW. This agreement serves not only as validation for the method of calculation, but also allows us to propose an alternate expression for PCE:

$$η = \frac{B Δ T + C Δ {T}^{2} - I ξ}{I - {I}_{tr}} \qquad Equation 2$$

where Iₜᵣ represents the transmitted power and I represents the reflection-corrected incident power, as usual. This new equation may be especially useful in situations where E_{λ} is unknown or cannot be easily measured, like during an in situ or in vitro experiment, provided transmitted light can be measured. It also allows determination of sample concentration for materials with known n, which may also be useful for determining the concentration of particles at tumor sites during in vitro photothermal conversion through the following relationship

$${E}_{λ} = log ( \frac{I - {I}_{tr}}{I ( 1 - ξ )} - 1 ) \qquad Equation 3$$

Finally, it is important to note that although a previous study has suggested that a substantial amount of residual cobalt may become trapped inside HGNs during shell formation, ICP-OES reveals that is not the case for our system. For the 0.20 OD solutions, gold concentrations were on the ppm level, with 5.0±0.9 μg/m L, 17.1±0.9 μg/m L, and 17.3±0.9 μg/L for HGN_{A-C}, respectively. Cobalt concentrations for the same samples were on the ppb level, with 200±100 μg/L, 210±80 μg/L, and 80±70 μg/L, respectively. Since the initial cobalt concentration for each sample exceeds 10,000 μg/L, this result indicates that virtually all residual cores oxidize into solution and ˜98% of initially present cobalt is removed during post-synthetic washing of the HGNs. Thus, we can be confident that the resultant optical and structural properties studied herein arise from the gold shells themselves and are not convoluted by the presence of residual cobalt.

In conclusion, HGN synthesis was modified to enable systematic variation in surface roughness and the effect of surface morphology on heat generation was investigated, providing the first quantification of the PCE of smooth and bumpy HGNs. Although bHGNs would be expected to have a larger scattering component than their smooth counterparts, the results herein show that they are capable of essentially equivalent heat generation, given equivalent OD. In theranostic applications, a bHGN may be more beneficial than a smooth HGN in a number of respects: in addition to maintaining excellent heat generation, the presence of bumps increases surface area for catalysis or drug loading and also contributes to local field enhancement for sensing or detection. A bHGN may also be more beneficial than solid spiky particles as the hollow core increases the effective surface area as compared to solid nanostructures. Overall, the present work further establishes the HGN as a highly customizable particle platform, offering tunable size, SPR, and now surface morphology with minimal and facile synthetic adjustment. The enhanced tunability of the HGN platform will enable optimized performance in applications spanning plasmonic photothermal therapy, imaging, sensing, and catalysis. More broadly, in establishing that bHGNs can also serve as excellent heat generators, our results have important implications for rational nanoparticle design, and lend support for other hollow bumpy structures to be considered as particularly powerful multimodal theranostic agents.

### Example 5—Calculation of Photothermal Conversion Efficiency

Photothermal conversion efficiency (PCE) was determined in accordance with the methods reported by Chen et al. (Small 2010, 6, 20, 2272-2280) and Roper et al. (J. Phys. Chem. C 2007, 111, 3636-3641). The energy balance of the system can be described by

$$( {m}_{S} {c}_{p , S} + {m}_{C} {c}_{p , C} ) \frac{d Δ T}{dt} = {Q}_{Laser} - {Q}_{Loss} \qquad Equation 4$$

where m_{S} is the mass of the solution, c_{p,S} is the heat capacity of the solution at constant pressure, m_{C} is the mass of the quartz cuvette, c_{p,C} is the heat capacity of the quartz cuvette, t is time, ΔT is the difference in temperature between time t and time 0, Q_{Laser} is the energy generated by laser illumination, and Q_{Loss} is the energy lost to the surroundings.

When the sample is illuminated, there are two contributions to Q_{Laser}: the energy due to light interaction with the particles (Q_{HGN}) and the energy due to light interaction with everything else, namely the cuvette walls and water solvent (Q_{OTHER}). Q_{HGN} may be described in terms of the heat dissipation due to electron-phonon relaxation of surface plasmons and Q_{OTHER} may be described as a fraction of incident energy. This can be written as

Q_{Laser}=Q_{HGN}+Q_{OTHER}=I(1−ξ)(1−10^{-E}^{_{λ}})η+Iξ Equation 5

where I is the reflection corrected incident laser power, ξ is the fraction of laser energy absorbed by the cuvette and water solvent, E_{λ} is the extinction at the illumination wavelength, and η is PCE.

Energy is dissipated to the surroundings through heat conduction and radiation from the surface. It may be modeled with a Taylor series as

Q_{Loss}=BΔT+C(ΔT)²+ Equation 6

where B and C are coefficients.

During steady state, Q_{Laser}=Q_{Loss}, which provides an equation for η according to

$$\begin{matrix}I ( 1 - ξ ) ( 1 - 1 {0}^{- {E}_{λ}} ) η + I ξ = B Δ T + {C ( Δ T )}^{2} & Equation 7 \\ η = \frac{B Δ T + C Δ {T}^{2} - I ξ}{I ( 1 - ξ ) ( 1 - {10}^{- {E}_{λ}} )} & Equation 8\end{matrix}$$

Thus, η may be calculated using Equation 8 once B, C, and ξ are determined. B and C are determined through temporal analysis of the cooling curves. During cooling, Q_{Laser}=0, and Equation 4 may be written as

$$( {m}_{S} {c}_{p , S} + {m}_{C} {c}_{p , C} ) \frac{d Δ T}{dt} = - B Δ T - {C ( Δ T )}^{2} \qquad Equation 9$$

where t_{c} is cooling time. Integrating ΔT with respect to t_{c} provides an exponential fit equation for the temperature decay. Letting

$${b}_{fit} = \frac{B}{a} and {c}_{fit} = \frac{c}{a} ,$$

where a=m_{S}c_{p,S}+m_{C}c_{p,C}, we may write

$$\frac{d Δ T}{{dt}_{c}} = - {b}_{fit} Δ T - {{c}_{fit} ( Δ T )}^{2} \qquad Equation 10$$

Now, Equation 10 may be integrated with respect to t_{c} in order to produce the following fit equation,

$$Δ T ( {t}_{c} ) = \frac{- {b}_{fit} {e}^{{b}_{fit} k}}{{c}_{fit} {e}^{{b}_{{fit}^{k}}} - {e}^{{b}_{{fit}^{{t}_{c}}}}} \qquad Equation 11$$

where k is a constant of integration. The cooling curve for HGN_{C} with E_{λ}=0.50 OD and its associated fit are shown in FIG. 5 as a representative example. The average B and C for our system were determined to be 2.00×10⁻² WK⁻¹ and 2.25×10⁻⁵ WK⁻², respectively. For ease of comparison with published studies reporting in minute timescales, this equates to 1.20 Jmin⁻¹K⁻¹ and 1.35×10⁻³ Jmin⁻¹K⁻², respectively. The Taylor series coefficients remained constant between samples and concentrations. This is expected as they are related to energy loss which should be a relatively consistent value for a given setup.

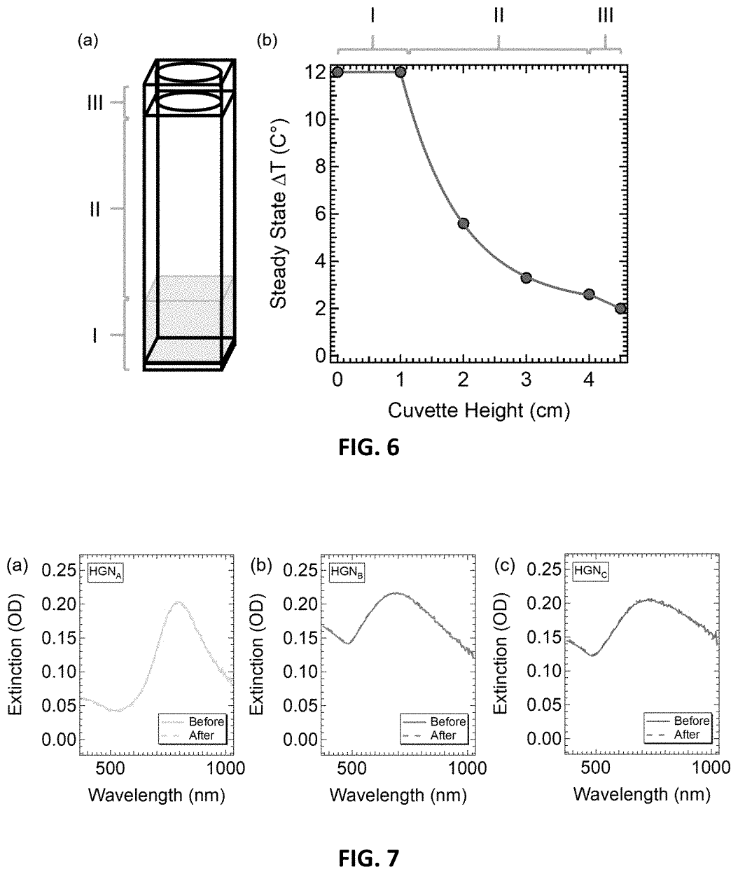

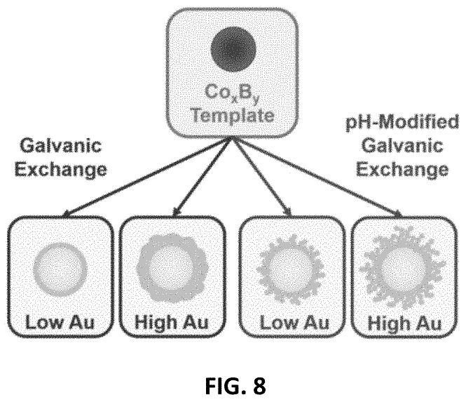

PCE values were determined according to Equation 8. Results are tabulated in Table 3. System parameters include m_{S}=1.000 g, c_{p,S}=4.184 J/gK, m_{C}=3.466 g, c_{p,C}=0.740 J/gK, I=0.345 W, and ξ=0.0171. It should be noted that m_{C} is the effective mass of the cuvette and involves a necessary correction due to the presence of a temperature gradient. For instance, when a 0.50 OD sample was at steady state with a 12° C. temperature increase, only the portion of the cuvette touching the solution was elevated by 12° C. Above this point, the cuvette temperature dropped steadily along the remaining height of the cuvette. A schematic is provided in FIG. 6, panel a to highlight three temperature regions of the cuvette and temperature measurements at steady state along the height of the cuvette are provided in terms of ΔT in FIG. 6, panel b.

TABLE 3
Cooling curve fit parameters and PCE calculation for
HGN_{A-C} at two concentrations (0.20 and 0.50 OD extinction).

| Sample | Average η (%) | E_{λ} (OD) | ΔT (K) | η (%) |
|---|---|---|---|---|
| HGN_{A} | 99 ± 1 | 0.20 | 6.5 | 98.2 |
| | | 0.50 | 11.9 | 99.8 |
| HGN_{B} | 96 ± 1 | 0.20 | 6.4 | 95.1 |
| | | 0.50 | 11.7 | 96.3 |
| HGN_{C} | 97 ± 1 | 0.20 | 6.4 | 96.6 |
| | | 0.50 | 11.7 | 97.6 |

The temperature gradient along the height of the cuvette affects the

$${m}_{C} {c}_{p , C} \frac{d Δ T}{dt}$$

term in Equation 4 since the entire mass of the cuvette was not elevated to the same final ΔT. To account for this temperature gradient, the ΔT curve in FIG. 5, panel b was integrated with respect to height. The cuvette volume was also determined as a function of height and converted to cuvette mass as a function of height using a cuvette density of 2.230 gcm⁻³. With the temperature and mass gradients determined, the m_{C}ΔT contribution could be calculated along the height of the cuvette and summed, resulting in a total m_{C}ΔT contribution of 41.60 g° C. Dividing this value by the original steady state ΔT gave an effective cuvette mass of 3.466 g. Without this correction, the PCE would be overestimated (an m_{C} that is falsely large would lead to an a that is falsely large, a B and C that are falsely large, and finally an η that is falsely large). No gradient was observed over the width of the cuvette walls.

### Example 6—Photostability of HGNs

The stability of HGN_{A-C} during heat generation experiments was confirmed by taking UV-Vis measurements before and after 30 minute exposure to the 1 W/cm² beam. Resultant extinction spectra are shown in FIG. 7. HGN_{A-C} retained their SPR shape and intensity after heat generation, demonstrating good stability during measurement. However, it should be noted that photostability may depend on laser power density, illumination time, and shell thickness.

### Example 7—Photothermal Conversion Efficiency Comparison to Other Nanosystems

The PCE values in Example 4 for smooth and bHGN are compared to those of previously reported Au nanostructures. PCE has also been determined for nanostructures of other materials, including other noble metals, metal sulfides, and related heterostructures. The previously reported PCE values for these nanosystems and a comparison to smooth and bHGN are provided in Table 4.

TABLE 4
Comparison of Photothermal Conversion Efficiencies. The SPR
column represents the reported λₘₐₓ. If no λₘₐₓ was reported,
NIR-I denotes SPR occurring in the first NIR window (~650-
950 nm) and NIR-II denotes SPR occurring in the second nm).
Vis denotes SPR occurring in the visible region (450-650 nm).

| | PCE | SPR | λ_{exc} | |
|---|---|---|---|---|
| Nanostructure | (%) | (nm) | (nm) | Reference |
| Cu₉S₅ Nanocrystal | 25.7 | Broad NIR-II | 980 | 4 |
| Au—Cu₂₋ₓSe Heterodimer | 32 | Broad NIR-II | 980 | 5 |
| AgS Nanodot | 35.0 | Broad Vis, | 795 | 6 |
| | | NIR-I | | |
| Hollow MoSx Nanoparticle | 39.6 | Broad NIR-I | 670 | 7 |
| Semiconducting Copolymer | 43.4 | Broad Vis, | 1064 | 8 |
| Nanoparticle | 44.9 | NIR-I, II | 808 | |
| Bi₂S₃ Nanoparticle | 51 | Broad Vis, | 808 | 9 |
| | | NIR-I | | |
| Pd Nanosheet | 52.0 | Broad NIR-I | 808 | 10 |
| Au Nanorod-Cu₇S₄ Dumbell | 55.8 | 809 | 808 | 11 |
| Multi-walled | 53 | Broad Vis, | 808 | 12 |
| Carbon Nanotubes | 49 | NIR-I, II | 980 | |
| | 56 | | 1090 | |
| Cu_{7.2}S₄ Nanocrystal | 56.7 | Broad NIR-II | 980 | 13 |
| Cu—Ag₂S Nanoparticle | 58.2 | Broad NIR-II | 808 | 14 |
| Au Nanorod-CuS Yolk-Shell | 61.34 | 980 | 980 | 15 |
| Au—Cu₇S₄ Core-Shell | 62.0 | 803 | 808 | 11 |
| Au—ZnS Core-Shell | 64 | 770 | 809 | 2 |
| Ag@Ag2S Core@Shell | 63.7 | Broad Vis, | 635 | 16 |
| Octopod | 64.7 | NIR-I, II | 808 | |
| | 79.3 | | 1064 | |
| Semiconducting Polymer | 71 | NIR-I | 808 | 17 |
| Nanoparticle with Vinylene | | | | |
| Bonds | | | | |
| Au—Ag₂S Core-Shell | 86 | 770 | 809 | 2 |
| Porous Pt | 89.9 | Broad Vis, | 980 | 18 |
| Nanoparticle | 97.0 | NIR-I, II | 809 | |
| bHGN | 96-97 | Broad NIR-I | 790 | This Study |
| Smooth HGN | 99 | 790 | 790 | This Study |

### Methods and Experimental

Synthesis of Cobalt-Based Scaffolds

Cobalt(II) chloride hexahydrate (CoCl₂.6H₂O) was purchased from Sigma-Aldrich, trisodium citrate dihydrate (Na₃C₆H₅O₇.2H₂O) was purchased from VWR International, and sodium borohydride (NaBH₄) was purchased from Fisher Scientific. All water used in synthesis was ultrapure in quality, with a resistivity of 18.3 MΩ.

CoₓB_{y} NP scaffolds were synthesized according to our previous report via the well-established nucleation of Co²⁺ ions with NaBH₄, using citrate as a capping ligand.¹⁴ Briefly, a 100 mL solution of 0.40 mM CoCl₂.6H₂O and 4.0 mM Na₃C₆H₅O₇.2H₂O was prepared in a 500 mL round-bottom flask and deaerated by bubbling with nitrogen for 1 hour. During this time, the solution was stirred at 700 rpm with a magnetic stir bar. Next, 120 μL of freshly prepared aqueous 1.0 M NaBH₄ nucleation agent gently mixed with 200 μL B(OH)₄⁻ growth agent was then injected into the stirring solution. The B(OH)₄⁻ agent was prepared in advance by hydrolyzing aqueous 1.0 M NaBH₄ in ambient conditions for 48 hours. After addition of the nucleation and growth agent mixture, the solution turned from pale pink to grey brown, indicating the nucleation of Co²⁺ ions and the formation of the CoₓB_{y} NP scaffold. After 2 minutes, the stir bar was magnetically suspended above the solution and the NPs were subsequently allowed to stand under a constant nitrogen flow for 2 hours to ensure complete hydrolysis of borohydride.

Dynamic Light Scattering

Dynamic light scattering (DLS) was performed on a DynaPro NanoStar from Wyatt Technology using Dynamics software version 7.1.7. Data acquisition parameters included water solvent, spherical radius of gyration (Rg) model, 20.000° C. temperature, and 30 acquisitions. The reported ± values represent one standard deviation from the mean. For measurement, 200 μL aliquots were extracted from the CoₓB_{y} solution under nitrogen protection and immediately transferred to the DLS instrument.

Synthesis of HGNs and bHGNs

Chloroauric acid (HAuCl₄) was purchased from Fisher Scientific. All water used in synthesis was ultrapure in quality, with a resistivity of 18.3 MΩ. Smooth HGNs were synthesized with anaerobic galvanic exchange, according to our previous report, with slight modification. Briefly, 4.0 μL of 0.10 M HAuCl₄ was added to 15 mL ultrapure water and deaerated by bubbling with nitrogen gas for 1 hour under magnetic stirring at 700 RPM. Once deaerated, galvanic exchange was initiated by transferring 15 mL of the CoₓB_{y} NP solution to the stirring gold solution via air-free cannula transfer. The resultant CoₓB_{y} NP/Au core/shell particles were stirred for 2 minutes under nitrogen protection at 700 rpm before final oxidation of the remaining CoₓB_{y} NP cores. Residual cores were fully oxidized by removing the septa and stirring at 700 rpm for an additional 3 minutes under ambient conditions. The resultant smooth HGNs were centrifuged at 1300 rpm for 3 minutes and resuspended in ultrapure water.

The bHGNs were synthesized with aerobic galvanic exchange. A given volume (8.0 μL or 40.0 μL) of 0.10 M HAuCl₄ was added to 15 mL ultrapure water and stirred at 700 rpm. The pH of the resultant gold solution was adjusted by addition of a given volume (4.0 μL, 10 μL, 20 μL, or 30 μL) of 1.0 M NaOH. The solution was allowed to equilibrate for 60 seconds while stirring. Equilibration time was adjusted to 1 hour as noted. Galvanic exchange was then initiated by transferring 15 mL of the CoₓB_{y} NP solution to the stirring pH-modified gold solution via cannula transfer. The resultant CoₓB_{y} NP+Au³⁺ solution was kept stirring for 5 minutes during which time galvanic exchange produced CoₓB_{y} NP/Au core/shell particles and oxidation in air removed the residual CoₓB_{y} core. The resultant bHGNs were centrifuged at 1300 rpm for 3 minutes and resuspended in ultrapure water.

UV-Vis Spectroscopy

UV-vis spectra were recorded with an Agilent Technologies Cary 60 UV-vis spectrophotometer using a 700 μL quartz cuvette with 10 mm optical path length. For CoₓB_{y} NP extinction measurement, 500 μL aliquots were extracted from the solutions under nitrogen protection and immediately transferred to the spectrophotometer.

Electron Microscopy

Scanning electron microscopy (SEM) was performed on an FEI Quanta 3D field emission microscope operated at 10.00 kV acceleration voltage. HGN solutions were dropped onto a hexagonal, 400 mesh copper grid with a carbon support film of standard 5-6 nm thickness (Electron Microscopy Sciences). High-resolution transmission electron microscopy (HRTEM) was performed on an FEI UT Tecnai microscope operated at 200 kV acceleration voltage. Diameter measurements were taken directly from SEM images using ImageJ software. For each sample, at least 100 HGN diameters were used to calculate the average diameter±one standard deviation. For rugose particles, outer diameter was measured from tip to tip.

PEGylation of HGNs and bHGNs

Heterobifunctional polyethylene glycol (PEG) functionalized with orthopyridyl disulfide (OPSS) and succinimidyl valerate (SVA) was purchased from Laysan Bio, Inc. For PEGylation, 100 μI of 1.0 mg/mL OPSS-PEG-SVA was added to 500 μL of 4.0 optical density (OD) HGN and shaken overnight. The resultant HGN-PEG solution was centrifuged once at 13,000 rpm for 3 minutes to remove residual PEG and resuspended in ultrapure water.

Evaluation of Photothermal Coupling

For assessment of photothermal conversion, 1.0 mL of HGN-PEG was placed in a quartz cuvette and exposed to a 790 nm NIR continuous wave laser with spot size of 7 mm and power density of 1.0 Wcm-2. The solution was stirred at 300 rpm over the course of the experiment. The laser was incident upon the sample for 30 minutes (heating cycle) and then blocked for 30 minutes (cooling cycling). The temperature of the solution was measured with a 33-gauge hypodermic thermocouple (Omega) and recorded with a data logger (Supco) at a rate of one readout per second. The thermocouple was placed directly into solution and the solution remained capped for the entirety of the measurement. For each sample, heat generation measurements were taken on two particle concentrations, corresponding to optical extinctions of 0.50 OD and 0.20 OD, as measured at 790 nm.

Inductively Coupled Plasma Optical Emission Spectrometry (ICP-OES)

ICP-OES was performed on a Thermo iCAP 7400 ICP-OES. Gold standards at 0 ppm (blank), 2 ppm, 5 ppm, 10 ppm, and 20 ppm were made using HAuCl₄ and ultrapure water with 5% v/v HCl. For sample preparation, 200 μL HGN was added to 1800 μL blank to create a 1:10 dilution. Internal standardization was carried out with Sc and Y internal references. Two Au wavelengths (242.795 nm and 267.595 nm) and two Co wavelengths (238.892 nm and 237.862 nm) were used for analysis.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of producing hollow metal nanospheres (HMNs) having a pre-selected surface rugosity, comprising:
combining in a galvanic exchange reaction at a selected pH:
a solution comprising cobalt-based nanoparticle (CoₓB_{y} NP) scaffolds; and
a solution comprising a metal,
to produce CoₓB_{y} NP core/metal shell structures; and
oxidizing the CoₓB_{y} NP cores of the CoₓB_{y} NP core/metal shell structures to produce HMNs having the pre-selected surface rugosity,
wherein the pH of the galvanic exchange reaction is selected to produce the pre-selected surface rugosity of the HMNs.

2. The method according to claim 1, wherein the pH of the solution comprising the metal is selected to produce the selected pH of the galvanic exchange reaction.

3. The method according to claim 2, wherein the selected pH of the solution comprising the metal is produced by combining a solution comprising the metal with a basic solution.

4. The method according to claim 3, wherein the basic solution is sodium hydroxide.

5. The method according to claim 1, wherein the galvanic exchange reaction is performed in an anaerobic environment.

6. The method according to claim 1, wherein the solution comprising the metal is deaerated prior to the combining with the solution comprising the CoₓB_{y} NP scaffolds.

7. The method according to claim 1, wherein the oxidizing is by oxygenation.

8. The method according to claim 1, wherein the HMNs are hollow gold nanospheres (HGNs).

9. The method according to claim 8, wherein the solution comprising the metal is chloroauric acid (HAuCl₄).

10. The method according to claim 9, wherein the pH of the HAuCl₄ is selected to produce the selected pH of the galvanic exchange reaction.

11. The method according to claim 10, wherein the selected pH of the HAuCl₄ is produced by combining HAuCl₄ with a basic solution.

12. The method according to claim 1, wherein the HMNs exhibit a surface plasmon resonance (SPR) absorption with a maximum peak position of from about 565 to about 1300 nm.

13. The method according to claim 1, further comprising, subsequent to producing the HMNs, attaching a targeting moiety to the surface thereof.

14. The method according to claim 13, wherein the targeting moiety binds to a molecule on the surface of a target cell.

15. Hollow metal nanospheres (HMNs) produced according to the method of claim 1.

16. A composition comprising the HMNs of claim 15.

17. A pharmaceutical composition, comprising:
the HMNs of claim 15; and
a pharmaceutically acceptable carrier.

18. A kit, comprising:
the HMNs of claim 15.

19. A method comprising administering to an individual in need thereof the HMNs of claim 15.

20. The method according to claim 19, wherein the individual in need thereof is in need of photothermal therapy (PTT).
